(19) United States
(12) Patent Application Publication
(10) Pub. No.: US 2024/0189800 A1
(43) Pub. Date: Jun. 13, 2024

(54) STABLE CATALYST FOR CARBON DIOXIDE TO METHANOL CONVERSION AND PROCESS OF CONVERSION THEREOF

(71) Applicant: COUNCIL OF SCIENTIFIC AND INDUSTRIAL RESEARCH, New Delhi (IN)

(72) Inventors: Pavan Naganath DONGAPURE, Pune (IN); Jyoti Rajesh TEKAWADIA, Pune (IN); Radhamonyammma Nandini DEVI, Pune (IN)

(73) Assignee: COUNCIL OF SCIENTIFIC AND INDUSTRIAL RESEARCH, New Delhi (IN)

(21) Appl. No.: 18/537,124

(22) Filed: Dec. 12, 2023

(30) Foreign Application Priority Data
Dec. 12, 2022 (IN) 202211071788

(51) Int. Cl. B01J 23/80; B01J 21/04; B01J 21/06; B01J 35/30; B01J 37/02; B01J 37/04; B01J 37/08; B01J 37/18; C07C 29/156

(52) U.S. Cl. CPC B01J 23/80; B01J 21/04; B01J 21/063; B01J 35/394; B01J 37/0215; B01J 37/0236; B01J 37/04; B01J 37/088; B01J 37/18; C07C 29/156

(57) ABSTRACT

The present disclosure is directed to a catalyst for carbon dioxide to methanol conversion comprising: 1-10 wt % of Nickel; 5-15 wt % of Zinc; and 80-95 wt % of metal oxide, wherein the wt % is based on the total weight of the catalyst. The present disclosure also provides a method for preparation of a catalyst for carbon dioxide to methanol conversion comprising: i) dissolving 1-10 wt % nickel precursor and 5-15 wt % zinc precursor in a solvent to obtain a first mixture; ii) adding 80-95 wt % metal oxide in the first mixture as obtained in step (a) to obtain a second mixture; iii) evaporating the solvent from the second mixture as obtained in step (b), drying and calcining in air at a temperature in the range of 350° C. to 500° C. for a period in the range of 2 hr to 4 hrs to obtain calcined mixture; and iv) reducing the calcined mixture as obtained in step (c) under hydrogen atmosphere at a temperature in the range of 500° C. to 700° ° C. for a period in the range of 1 hr to 3 hrs to obtain the catalyst. The disclosure also provides a low pressure process for conversion of carbon dioxide to methanol.

### FIELD OF THE INVENTION

The present invention relates to a stable catalyst for carbon dioxide to methanol conversion and a process for the preparation of the catalyst. Particularly, present invention relates to conversion of carbon dioxide to methanol. More particularly, present invention relates to a low-pressure process for conversion of carbon dioxide to methanol.

### BACKGROUND OF THE INVENTION

Climate change and global warming are the major issues in today's scenario which affects not just the human community but other living beings on the planet as well. Uncontrolled emissions of CO₂ (carbon dioxide) from various sources into the atmosphere has led to a rapid increase in average global temperature. Many techniques have been developed for reducing the CO₂ level in atmosphere but they have not been enough and the steady rise of CO₂ has now reached almost 415 ppm. Thus, it is imperative to not just reduce the generation of CO₂ but also capture the CO₂ already released into the atmosphere.

Capture and conversion of CO₂ to industrially useful compounds or fuels such as methanol is very effective in reducing atmospheric CO₂. Methanol is one of the key chemicals useful for the synthesis of acetic acid, DME, formaldehyde etc. which are used in products like plywood subfloors, solvents, windshields and adhesives. Methanol is prepared from syngas (mixture of hydrogen and carbon monoxide), from biomass/municipal waste and now also by reduction of CO₂. However, use of CO₂ is an energy intensive process, with ΔG298K°=−394.4 kJ mol-1 giving it an inherent property of inertness. Therefore, catalysts play a very important role in reduction of CO₂. Global capacity to produce methanol is approximately 157 million metric ton and is expected to double by 2030. Synthesizing very efficient and durable catalyst for this reduction of CO₂ to methanol can help control the carbon dioxide crisis and contribute to methanol industry.

Many different catalysts have been developed and tested but they are either unstable or work under high pressures for reasonable conversions. Currently, in industries Cu based catalysts are used for methanol synthesis from CO₂/CO that works under high pressures.

Reference may be made to Journal “Discovery of a Ni—Ga catalyst for carbon dioxide reduction to methanol” by Jens K. Nørskov et al. [Nature Chemistry 2014, 6, 320-324] showed intermetallic catalyst of Ni—Ga can activate CO₂ and reduce to methanol at ambient pressure.

Reference may be made to Journal “Journal of CO2 Utilization Volume 39, July 2020, 101151” reports the preparation of Ni—Ga and M-Ni—Ga (M=Au, Co, Cu) catalysts supported over silica (loading of M-Ni—Ga of 10 wt %) by wetness impregnation, followed by drying at 100° C. and reduction at 680° C., and their evaluation for methanol synthesis using 1:3 CO₂:H₂ mixture at 10 bar and 200-270° C. However, the catalyst requires the expensive element Ga which is not readily available and not preferable.

Thus, there is an urgent need in the art to provide for a catalyst and a process for CO₂ conversion to methanol that is efficient, cost-effective and can be performed at low pressure.

### OBJECTS OF THE INVENTION

Main object of the present invention is to provide a catalyst for conversion of carbon dioxide to methanol that is stable and cost-effective.

Another object of the present disclosure is to provide a method for preparation of a catalyst for carbon dioxide to methanol conversion.

Yet, another object of the present disclosure is to provide a low pressure process for conversion of carbon dioxide to methanol.

### SUMMARY OF THE INVENTION

The present invention relates to a stable catalyst and a process for synthesizing methanol from the environmental pollutant carbon dioxide which allows operation at low pressures.

Accordingly, the present invention provides a catalyst for carbon dioxide to methanol conversion comprising:
a) 1-10 wt % of a Nickel metal of the total weight of the catalyst;
b) 5-15 wt % of a Zinc metal of the total weight of the catalyst; and
c) 80-95 wt % of a metal oxide of the total weight of the catalyst.

In an embodiment of the present invention, the catalyst comprises Ni metal in a range of 1-10 wt %, preferably in the range of 1-3 wt %.

In another embodiment of the present invention, the catalyst comprises Zn metal in a range of 5-15 wt %, preferably in the range of 7-9 wt %.

In yet another embodiment of the present invention, the catalyst comprises titania in a range of 80-95 wt %, preferably in the range of 87-92 wt %.

In yet another embodiment of the present invention, the metal oxide is selected from a group consisting of titania, zinc oxide, alumina or combinations thereof.

In yet another embodiment of the present invention, the metal oxide is preferably titania.

In yet another embodiment of the present invention, the titania may be in any of the phases brookite, rutile, anatase or combinations thereof.

In yet another embodiment of the present invention, the titania is most preferably P25 titania which is a mixture of rutile and anatase titania.

In yet another embodiment of the present invention, the catalyst comprises a metal loading of 10% and 90% support. The catalyst comprises 2 wt % nickel and 8 wt % zinc supported on titania P25.

In yet another embodiment of the present invention, the catalyst is a catalyst system comprising NiZn alloy with 50:50 wt % ratio supported on anatase and rutile phases of TiO₂ or ZnO.

In yet another embodiment of the present invention, ZnO may be present in trace amounts.

In yet another embodiment of the present invention, the catalyst may be synthesized by wet impregnation method.

In yet another embodiment, the present invention provides a method for preparation of a catalyst for carbon dioxide to methanol conversion comprising the steps of:
i) dissolving 1-10 wt % nickel precursor and 5-15 wt % zinc precursor in a solvent to obtain a first mixture;
ii) adding 80-95 wt % metal oxide in the first mixture as obtained in step (a) to obtain a second mixture;
iii) evaporating the solvent from the second mixture as obtained in step (b), drying and calcining in air at a temperature in the range of 350° C. to 500° C. for a period in the range of 2 hr to 4 hrs to obtain calcined mixture; and
iv) reducing the calcined mixture as obtained in step (c) under hydrogen atmosphere at a temperature in the range of 500° C. to 700° ° C. for a period in the range of 1 hr to 3 hrs to obtain the catalyst.

In yet another embodiment of the present invention, the nickel precursor is selected from a group consisting of nickel nitrate, nickel sulfate, nickel chloride, their hydrates, or combinations thereof.

In yet another embodiment of the present invention, the nickel precursor is nickel nitrate (II) hexahydrate.

In yet another embodiment of the present invention, the zinc precursor is selected from a group consisting of zinc nitrate, zinc sulfate, zinc chloride, their hydrates, or combinations thereof.

In yet another embodiment of the present invention, the zinc precursor is zinc nitrate (II) hexahydrate.

In yet another embodiment of the present invention, the solvent is selected from a group consisting of water, ethanol, methanol and combination thereof.

In yet another embodiment of the present invention, calcination may be performed at 450° C. for 3 hours.

In yet another embodiment of the present invention, the reduction is performed at 550° C. for 2 hours.

In yet another embodiment of the present invention, the air and hydrogen have a flow rate in the range of 20-25 ml/min.

In yet another embodiment, the present invention provides a method of carbon dioxide to methanol conversion using the catalyst as defined above comprising the steps of:
(A) contacting carbon dioxide with the catalyst as defined above under atmosphere of hydrogen and nitrogen at a temperature in the range of 200-300° C. for a period in the range of 1 to 5 hrs with a pressure in the range of 2 to 40 bars to obtain methanol.

In yet another embodiment of the present invention, the atmosphere of hydrogen and nitrogen have a flow rate in a range of 10 mL/min to 500 mL/min and gas hourly space velocity (GHSV) in the range of 1000 h⁻¹ to 15000 h⁻¹.

In yet another embodiment of the present invention, the carbon dioxide to hydrogen ratio is 1:3.

### BRIEF DESCRIPTION OF THE DRAWINGS

FIG. 1 provides high resolution transmission electron micrographs (HR-TEM) of the catalysts reduced at 550° C. (a is for 5 nm and b is for 50 nm), 600° C. (c is for 5 nm and d is for 50 nm) and 650° ° C. (e is for 5 nm and f is for 50 nm).

FIG. 2 provides the x-ray diffraction patterns of:
(a) a—catalyst reduced at 550° C., and b-catalyst when used in CO₂ conversion process; (b) a-catalyst reduced at 600° C., and b—catalyst when used in CO₂ conversion process; (c) a—catalyst reduced at 650° C., and b—catalyst when used in CO₂ conversion process, wherein the catalyst is as per an exemplary embodiment of the present disclosure. Standard simulated patterns of Ni—Zn alloy and ZnO are given for comparison.

FIG. 3 compared the methanol space time yield as a function of amount of methanol generated per gm of a catalyst per hour in the process of conversion of carbon dioxide to methanol with reduction performed at pressures ranging from 2.5 bars to 25 bars where the catalyst is:
a) 2% Ni-8% Zn supported by TiO₂ reduced at 550° ° C.;
b) 2% Ni-8% Zn supported by TiO₂ reduced at 600° C.; and
c) 2% Ni-8% Zn supported by TiO₂ reduced at 650° C.

FIG. 4 shows selectivity of the catalysts at 2.2, 20 and 40 bar.

FIG. 5 shows the in-situ Diffuse Reflectance Infrared Fourier Transform Spectroscopy (DRIFT) spectra of catalyst surface of 2% Ni-8% Zn supported by TiO₂ and reduced at 650° C. while passing only CO₂ (5 ml/min) over it at atmospheric pressure.

FIG. 6 depicts the durability of a catalyst reduced at 550° C. in generating methanol per gm of the catalyst over a period of 36 h under varying pressure and temperature conditions.

### DETAILED DESCRIPTION OF THE INVENTION

The present disclosure provides an efficient catalyst for capture and conversion of carbon dioxide to methanol for suitable industrial applications. The disclosure also provides a process for conversion of carbon dioxide to methanol at low pressures employing the catalyst.

The present disclosure provides a stable catalyst for carbon dioxide to methanol conversion comprising:
a) 1-10 wt % of a Nickel metal of the total weight of the catalyst;
b) 5-15 wt % of a Zinc metal of the total weight of the catalyst; and
c) 80-95 wt % of a metal oxide of the total weight of the catalyst.

The metal oxide is selected from a group consisting of titania, zinc oxide, alumina and combination thereof.

The catalyst is an intermetallic catalyst comprising metals including nickel and zinc. The metals are supported on a metal oxide preferably titania. Thus, the catalyst comprises nickel and zinc alloy supported on titania. The catalyst comprises an interface of nickel and zinc alloy with zinc oxide and titania.

The present invention provides a method for preparation of a catalyst for carbon dioxide to methanol conversion comprising:
i) dissolving 1-10 wt % nickel precursor and 5-15 wt % zinc precursor in a solvent to obtain a first mixture;
ii) adding 80-95 wt % metal oxide in the first mixture as obtained in step (a) to obtain a second mixture;
iii) evaporating the solvent from the second mixture as obtained in step (b), drying and calcining in air at a temperature in the range of 350° ° C. to 500° C. for a period in the range of 2 hr to 4 hrs to obtain calcined mixture; and
iv) reducing the calcined mixture as obtained in step (c) under hydrogen atmosphere at a temperature in the range of 500° ° C. to 700° C. for a period in the range of 1 hr to 3 hrs to obtain the catalyst.

The solvent may be any well-known solvent in the art capable of dissolving the nickel precursor, zinc precursor and titania, including but not limited to, water, ethanol, methanol and the like.

The solvent is selected from a group consisting of water, ethanol, methanol and combination thereof. Preferably, the solvent is water.

The nickel precursor is selected from a group consisting of nickel nitrate, nickel sulfate, nickel chloride, their hydrates, or combinations thereof.

The zinc precursor is selected from a group consisting of zinc nitrate, zinc sulfate, zinc chloride, their hydrates, or combinations thereof.

The present invention provides a method of carbon dioxide to methanol conversion comprising:
(A) contacting carbon dioxide with the catalyst as claimed in claim 1 under atmosphere of hydrogen and nitrogen at a temperature in the range of 200-300° C. for a period in the range of 1 to 5 hrs with a pressure in the range of 2 to 40 bars to obtain methanol.

The carbon dioxide is contacted with the catalyst at a temperature in the range of 200-300ºC, preferably at 250ºC for a period in the range of 1 to 5 hrs, preferably for 2-3 hrs with a pressure in the range of 2 to 40 bars.

The atmosphere of hydrogen and nitrogen have a flow rate in a range of 10 ml/min to 500 mL/min, preferably 50 mL/min 500 mL/min, more preferably 100 mL/min.

The atmosphere of hydrogen and nitrogen may have a gas hourly space velocity (GHSV) of 1000 h⁻¹ to 15000 h⁻¹, preferably 9000 h⁻¹.

The carbon dioxide to hydrogen ratio is 1:3.

The present disclosure provides a low pressure process for conversion of carbon dioxide to methanol, the process comprising the step of contacting carbon dioxide with a catalyst comprising Ni—Zn supported on metal oxide under an atmosphere of hydrogen and nitrogen.

The process is performed on a fixed or moving bed reactor.

The process has yield methanol in a range of 0.005 g/g of catalyst/hour to 0.030 g/g of catalyst/hour, preferably in a range of 0.015 g/g of catalyst/hour to 0.020 g/g of catalyst/hour.

The process is combined with another process that employs methanol and generates carbon dioxide to give a cyclic process.

The catalyst comprises Ni—Zn bimetals supported on TiO₂ support prepared by wet impregnation.

The catalyst comprises alloy of Ni—Zn or bimetallic system of Ni and Zn especially with ZnO with rich regions in the close proximity as the active catalyst wherein said alloy of Ni—Zn or bimetallic system of Ni and Zn is optionally or specifically supported on a support.

Carbon dioxide is obtained through any suitable source such as from atmosphere, carbon dioxide obtained as by product from industrial sources such as power plants, and the like. The carbon dioxide is captured from the atmosphere by the catalyst.

The source of hydrogen includes a variety of sources such as hydrogen obtained from splitting of water, atmosphere, and the like.

Methanol produced by the above process is employed as an alternative environment friendly fuel. It may be used in the manufacture of chemicals including dimethyl ether, hydrogen, acetic acid, styrene, or formaldehyde which may be further employed in adhesives, construction materials, resins and the like.

The present invention (catalyst and process) is not limited to methanol production only, but also include other oxygenated products formation such as higher alcohols having carbon atoms up to 10 (C1-C10, e.g. ethanol, propanol, butanol, pentanol, hexanol, etc.), aldehydes (formaldehyde, etc.), acid (e.g. acetic acid, formic acid, etc), ether (DME—dimethyl ether, etc.) and so on, based on substrate/reagents keeping CO₂ and catalyst the same as mentioned above.

Nickel and zinc surfaces are generally active for CO₂ and hydrogen activation. However, individually these elements either have no activity for the reduction or cause methanation of carbon dioxide. The catalyst is capable of capturing carbon dioxide from atmosphere. The components of the catalyst synergistically enable the conversion of carbon dioxide to methanol at low pressures. Conventionally intermetallic Ni—Zn catalysts employed in conversion of carbon dioxide would yield methane, but the synergism of the titania and the metals minimize methane from coming out of the catalyst system, driving the formation of methanol with more selectivity.

The catalyst of the present disclosure does not require high pressures of 50-100 bars for the reduction of carbon dioxide and performs efficient reduction with good yields at low pressures including 2-40 bars. Methanol formation in low concentration is also observed even at 2 bars. The reduction in pressures decreases the manufacturing costs of methanol from carbon dioxide.

### EXAMPLES

Following examples are given as a way of illustration only and should not be construed to limit the scope of the present invention.

### Details of the Chemicals Used

The following chemicals, gases and materials were used:
nickel(II) nitrate hexahydrate (Ni(NO₃)₂·6H₂O), 99.999%, Sigma-Aldrich);
zinc(II) nitrate hexahydrate (Zn(NO₃)₂·6H₂O, 98%, Sigma-Aldrich);
CO₂ (99.99%, EffecTech);
H₂ (99.998%, Sandesh Gases); and
N2 (99.998%, Sandesh Gases).

### Example 1: Synthesis of Intermetallic Catalyst [2% Ni-8% Zn/90% TiO₂ Catalyst]

2% Ni-8% Zn/90% TiO₂ intermetallic catalyst was prepared by wet impregnation. Precursor salts, Nickel(II) nitrate hexahydrate (0.198 g) and Zinc(II) nitrate hexahydrate (0.7279 g) were dissolved in 30-40 ml of water and stirred with TiO₂ (P25: titania which is a mixture of rutile and anatase titania) (1.5 g) for 2 hrs at a temperature range of 25-30° C. Water was removed by rotary evaporator, dried overnight (about 10 hrs) in oven at 100° C. and calcined in air in a U-Tube furnace at 450° C. for 3 hrs under air flow with rate of 20-25 ml/min for 3 hrs with ramping rate 3° C./min. The catalyst was then divided into three batches which were reduced at 550° C., 600° C., and 650° C. respectively for 2 hrs, in the presence of H₂ with rate of 20-25 ml/min.

### Example 2: Synthesis of Intermetallic Catalyst: [2% Ni-8% Zn/90% Al₂O₃ Catalyst]

2% Ni-8% Zn/90% Al₂O₃ intermetallic catalyst was prepared by wet impregnation. Precursor salts, Nickel(II) nitrate hexahydrate (0.198 g) and Zinc(II) nitrate hexahydrate (0.7279 g) were dissolved in 30-40 ml of water and stirred with Al₂O₃ (1.5 g) for 2 hrs at a temperature range of 25-30° C. Water was removed by rotary evaporator, dried overnight (about 10 hrs) in oven at 100° C. and calcined in air in a U-Tube furnace at 450° C. for a period in the range of 3 hrs under air flow with rate of 20-25 ml/min for 3 hrs with ramping rate 3ºC/min. the catalyst was then reduced at 550° ° C. for 2 hrs while passing H₂ with rate of 20-25 ml/min.

### Example 3: Characterization of Intermetallic Catalyst

High resolution transmission electron micrographs (HR-TEM) images were collected using an FEI HRTEM instrument at 300 kV. Samples were prepared by dispersing in ethanol and drop casting on a holey C Cu grid. This was dried overnight in air before analyzing.

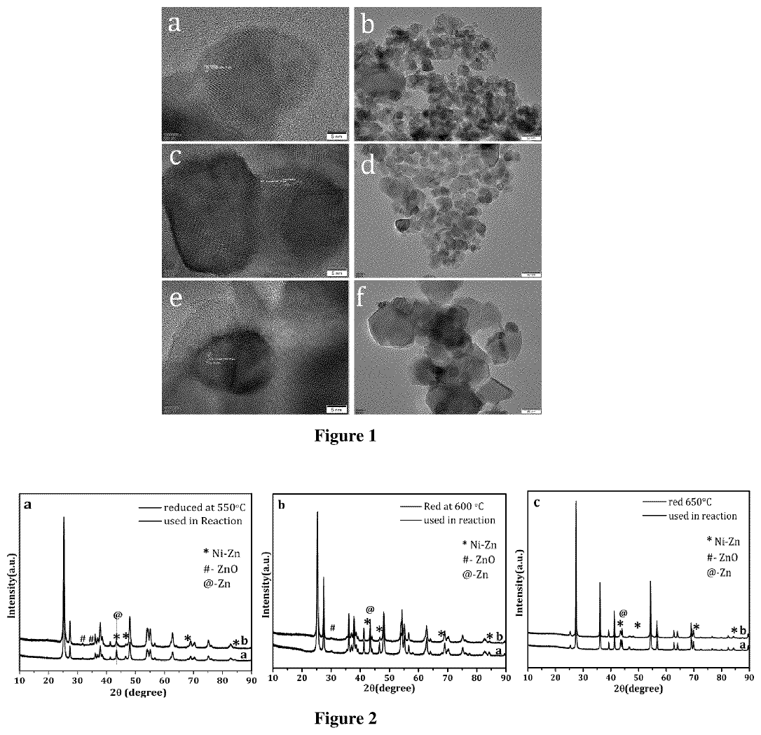

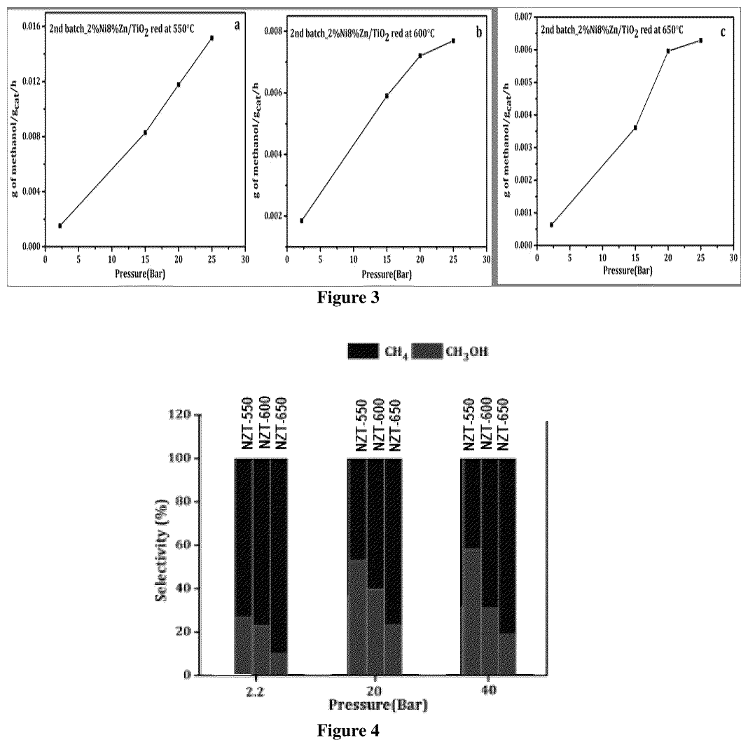

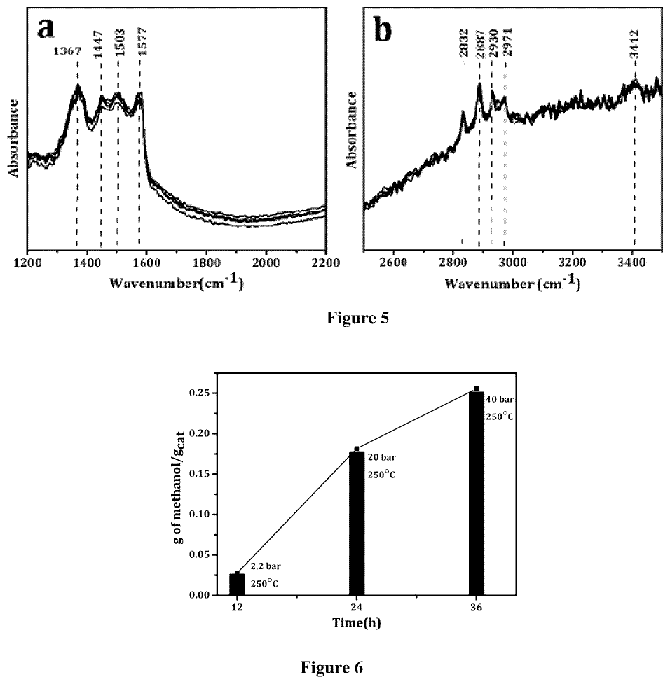

FIG. 1 provides HR-TEM of the catalysts reduced at 550° C. (a is 5 nm resolution image and b is 50 nm resolution image), 600° C. (c is 5 nm resolution image and d is 50 nm resolution image) and 650° C. (e is 5 nm resolution image and f is 50 nm resolution image).

XRD patterns were recorded in a PANalyticalX′Pert Pro dual goniometer diffractometer with Ni as filter and Cu Kα source operating at 40 kV and 30 mA with step size 0.0083° and time per step 91.44 s. X'celerator solid state detector was used for recording the data.

FIG. 2 provides the X-ray diffraction patterns of: (a) a—catalyst reduced at 550° C., and b-catalyst when used in CO₂ conversion process; (b) a—catalyst reduced at 600° C., and b—catalyst when used in CO₂ conversion process; and (c) a—catalyst reduced at 650° ° C., and b—catalyst when used in CO₂ conversion process.

HR-TEM shows the Ni—Zn inter-metallic particles were available on the surface of TiO₂ in all reduced samples and powder XRD analysis shows that d—spacing also matched with those of Ni—Zn. XRD analysis clearly shows the presence of Ni—Zn intermetallics were formed at reduced temperatures around 43.30 (2θ). Catalyst which was reduced at 550° C. showed ZnO impurities with peaks around 31.8 and 34.50 (2θ), it may due to lesser reduction temperature as compared to other two samples which were reduced at 600° ° C. and 650° C. At 600° C., ZnO peak intensity reduced, at the same time titania phase started to change from anatase to rutile but did not change completely thus the catalyst has Ni—Zn alloy on TiO₂ with anatase-rutile mixture. Also, at 600° C., ZnO was absent and the presence of the new phase of Zn₂Ti₃O₈ was confirmed from HRTEM and SAED patterns; HAADF imaging indicated an increase in particle size of NiZn particles to ˜75 nm; and EDS mapping and line profile of a single NiZn particle clearly showed the Zn and Ti rich domains corroborating an interface with Zn₂Ti₃O₈ phase. At 650° C., there was total reduction of ZnO peak and peaks related to Ni—Zn alloy along with phase change of titania to rutile. Surprisingly, at 650° ° C., neither ZnO nor Zn₂Ti₃O₈ was observed; Zn based oxides may have vaporized at the high temperature without forming the stable Zn titanate. Unexpectedly, the size of Ni—Zn particles was smaller than NZT-600 but almost similar to NZT-550. From the elemental quantification by EDS analysis of all the samples, the Ni atomic fraction (%) was found to be in the range of 50-55, and the atomic fraction (%) of Zn varies between 45-50.

From HAADF and EDS mapping, it becomes clear that NiZn particles of size ˜40 nm (particle size distribution) are well dispersed on the TiO₂ surface. Interestingly, almost all the NiZn particles are in the close vicinity of ZnO particles, projecting a clear interface between NiZn and ZnO nanoparticles. Oxygen rich layer around NiZn particle could have formed due to atmospheric surface oxidation.

### Example 4: Conversion of Carbon Dioxide to Methanol

2% Ni-8% Zn/90% TiO₂ catalyst synthesized in Example 1 was tested for the conversion of carbon dioxide to methanol. The CO₂ reduction was performed at 250° C. with pressure varying from 2.2 20, and 40 bar employing catalysts: a) 2% Ni-8% Zn supported by TiO₂ reduced at 550° C.; b) 2% Ni-8% Zn supported by TiO₂ reduced at 600° C.; and c) 2% Ni-8% Zn supported by TiO₂ reduced at 650° C. A downflow fixed bed reactor was loaded with 0.4 cc of the catalyst. CO₂, N₂ and H₂ flow rates were controlled by mass flow controllers and the reactor was fitted with high pressure valves and connections. Ratio of the CO₂ to hydrogen was controlled to 1:3 with total flow rate of 60 mL/min and GHSV of 9000 h⁻¹. The catalyst was pretreated under H₂ gas for 2 h at 250° C. and then switched to the reactant feed. The outlet of the reactor was connected to a series of bubblers with water and analyzed by HPLC. The gas separated was analyzed online by GC. The methanol space time yield as a function of amount of methanol generated per gm of the catalyst per hour for the different catalysts and process conditions is presented in Table 1.

TABLE 1
Methanol yield of 2% Ni-8% Zn/TiO₂ and 4% Ni-8% Zn/TiO2 catalyst
reduced at different temperature/pressure conditions

| | Catalyst reduced | Catalyst reduced | Catalyst reduced | 4% Ni-8% Zn/TiO2 |
|---|---|---|---|---|
| | at 550° C. | at 600° C. | at 650° C. | Reduced at 550° C. |
| Reaction | (Methanol STY) | (Methanol STY) | (Methanol STY) | Methanol STY) |
| pressure | g of | g of | g of | g of |
| (Bar)/T (° C.) | methanol/g_{cat}/h | methanol/g_{cat}/h | methanol/g_{cat}/h | methanol/g_{cat}/h |
| 2.2/250 | 0.00229 | 0.00520 | 0.00120 | 0.000701 |
| 20/250 | 0.01512 | 0.01242 | 0.00606 | 0.004224 |
| 40/250 | 0.02128 | 0.01552 | 0.00829 | 0.005315 |

*STY—space time yield.

Interestingly, the catalysts which are reduced at higher temperatures, viz., 550, 600 and 650° C., exhibited methanol formation at near ambient pressures (2.2 bar), but the catalysts reduced at lower temperatures (300 and 400° C.) did not show any methanol formation at near ambient pressures.

FIG. 3 shows the methanol space time yield for all three catalysts. Among these catalysts, 2% Ni-8% Zn supported by TiO₂ and reduced at 550° C. exhibited most activity. It may due to more effective CO₂ adsorption on its titania surface compared to other catalysts and appropriate Ni—Zn particle size, and its interface with very small particles of ZnO.

FIG. 4 shows the selectivity of the catalysts at 2.2, 20 and 40 bar for methanol and methane which is the only by-product obtained. At near ambient pressure, selectivity is more for methane, but as pressure increases, NZT-550 shows more selectivity towards methanol.

FIG. 5 shows the in-situ infra-red spectra collected while passing only CO₂ (5 ml/min) over reduced catalyst surface of 2% Ni-8% Zn supported by TiO₂ and reduced at 650° C. at atmospheric pressure. The bands at 1367 cm⁻¹ and 1577 cm⁻¹ are assigned to symmetric and asymmetric vibrations of formate, respectively, and corresponding peaks of C—H stretching of formate are present at 2887 cm⁻¹ and 2971 cm-1, respectively. The bands at 2832 cm⁻¹, 2930 cm⁻¹ and 3412 cm⁻¹ are ascribed to adsorbed methanolic species on the catalytic surface. The peaks at 1447 cm⁻¹ and 1503 cm⁻¹ are assigned to carbonate species.

The yield of methanol generated was compared for catalyst with different supports such as silica, alumina and indium oxide. Results are presented in Table 2. Thus, it is clear that not all supports generate methanol from carbon dioxide.

TABLE 2
Comparison of Ni—Zn supported on different supports

| | 2% Ni-8% | | 2% Ni-8% | | |
|---|---|---|---|---|---|
| | Zn/TiO₂ | 2% Ni-8% | Zn/TiO₂ | 2% Ni-8% | 2% Ni-8% |
| Reaction | (anatase) | Zn/SiO₂ | (p-25)-Al₂O₃ | Zn/Al₂O₃ | Zn/In₂O₃ |
| pressure | g of | g of | g of | g of | g of |
| (Bar)/T | methanol/ | methanol/ | methanol/ | methanol/ | methanol/ |
| (° C.) | g_{cat}/h | g_{cat}/h | g_{cat}/h | g_{cat}/h | g_{cat}/h |
| 2.2/250 | — | — | 0.00049 | 0.00083 | — |
| 20/250 | — | — | 0.003903 | 0.003782 | — |
| 40/250 | — | — | 0.00549 | 0.005089 | — |

### Example 5: Durability of the Catalyst

The durability for the catalyst reduced at 550° ° C. was studied for up to 36 hours under varying conditions. After placing the catalyst in the reactor, the temperature was set to 250° C. and the pressure was increased to 2.2 bars and the reaction was monitored for 12 h in the same condition. After 12 h the sample was collected and analyzed. Again, the pressure was increased to 20 bars and 40 bars in 12 h intervals and the collected sample was analyzed. The catalyst was found to be stable across all the conditions (FIG. 6).

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Advantages of the Invention

The present disclosure provides a stable and efficient catalyst for conversion of carbon dioxide to methanol.

The present disclosure provides a facile and low pressure process for conversion of carbon dioxide to methanol.

The present disclosure provides a method of mitigating the effects of high atmospheric carbon dioxide to produce industrially useful methanol.

We claim:

1. A catalyst for carbon dioxide to methanol conversion comprising:
a) 1-10 wt % of a Nickel metal of the total weight of the catalyst;
b) 5-15 wt % of a Zinc metal of the total weight of the catalyst; and
c) 80-95 wt % of a metal oxide of the total weight of the catalyst.

2. The catalyst as claimed in claim 1, wherein the metal oxide is selected from a group consisting of titania, zinc oxide, alumina or combinations thereof.

3. A method for preparation of a catalyst for carbon dioxide to methanol conversion comprising the steps of:
i) dissolving 1-10 wt % nickel precursor and 5-15 wt % zinc precursor in a solvent to obtain a first mixture;
ii) adding 80-95 wt % metal oxide in the first mixture as obtained in step (a) to obtain a second mixture;
iii) evaporating the solvent from the second mixture as obtained in step (b), drying and calcining in air at a temperature in the range of 350° C. to 500° C. for a period in the range of 2 hr to 4 hrs to obtain calcined mixture; and
iv) reducing the calcined mixture as obtained in step (c) under hydrogen atmosphere at a temperature in the range of 500° ° C. to 700° C. for a period in the range of 1 hr to 3 hrs to obtain the catalyst.

4. The method as claimed in claim 3, wherein the solvent is selected from a group consisting of water, ethanol, methanol and combination thereof.

5. The method as claimed in claim 3, wherein the nickel precursor is selected from a group consisting of nickel nitrate, nickel sulfate, nickel chloride, their hydrates, or combinations thereof.

6. The method as claimed in claim 3, wherein the zinc precursor is selected from a group consisting of zinc nitrate, zinc sulfate, zinc chloride, their hydrates, or combinations thereof.

7. The method as claimed in claim 3, wherein the air and hydrogen have a flow rate in the range of 20-25 ml/min.

8. A method of carbon dioxide to methanol conversion using the catalyst as claimed in claim 1 comprising the steps of:
i. contacting carbon dioxide with the catalyst as claimed in claim 1 under atmosphere of hydrogen and nitrogen at a temperature in the range of 200-300° C. for a period in the range of 1 to 5 hrs with a pressure in the range of 2 to 40 bars to obtain methanol.

9. The method as claimed in claim 8, wherein the atmosphere of hydrogen and nitrogen have a flow rate in a range of 10 mL/min to 500 mL/min and gas hourly space velocity (GHSV) in the range of 1000 h⁻¹ to 15000 h⁻¹.

10. The method as claimed in claim 8, wherein the carbon dioxide to hydrogen ratio is 1:3.
